(19) United States
(12) Patent Application Publication
(10) Pub. No.: US 2021/0346685 A1
(43) Pub. Date: Nov. 11, 2021

(54) Catheter System for Explanting an Intracardiac Medical Device, Particularly a Pacing System

(71) Applicant: BIOTRONIK SE & Co. KG, Berlin (DE)

(72) Inventors: Brian M. TAFF, PORTLAND, OR (US); Nicholas DEVICH, TUALATIN, OR (US)

(73) Assignee: BIOTRONIK SE & Co. KG, Berlin (DE)

(21) Appl. No.: 17/286,199

(22) PCT Filed: Sep. 19, 2019
(86) PCT No.: PCT/EP2019/075130

§ 371 Date: Apr. 16, 2021

Related U.S. Application Data
(60) Provisional application No. 62/746,569, filed on Oct. 17, 2018.

(51) Int. Cl. A61N 1/05; A61B 17/3205; A61M 25/00

(52) U.S. Cl. CPC A61N 1/057; A61B 17/32056; A61N 1/3756; A61M 25/0074; A61M 25/0082

(57) ABSTRACT

A catheter system for explanting an intracardiac medical device, comprising: a catheter having a distal catheter tip configured to surround the intracardiac medical device and an encapsulation tissue that has grown around the intracardiac medical device, and a deployable cutting element at the distal catheter tip.

### CROSS-REFERENCE TO RELATED APPLICATIONS

This application is the United States national phase under 35 U.S.C. § 371 of PCT International Patent Application No. PCT/EP2019/075130, filed on Sep. 19, 2019, which claims the benefit of U.S. Patent Application No. 62/746,569, filed on Oct. 17, 2018, the disclosures of which are hereby incorporated by reference herein in their entireties.

### TECHNICAL FIELD

The present invention relates to a catheter system for explanting an intracardiac medical device.

Particularly, the disclosure relates to a cutting-tip-enabled catheter system for explanting an anatomically-encapsulated intracardiac medical device, particularly an intracardiac pacing system, e.g. a leadless pacemaker.

### BACKGROUND

Present market offerings for bradycardia support are increasingly pointing attention to the reduced, overall patient care risk profile touted by leadless pacemaker systems as compared to traditional, pocket-based formats. This revised support modality employs small, self-contained pulse generators that are anchored (e.g. via tine-based structures) within the heart's blood volume to administer therapy. Such designs have, thus far, leaned on the use of primary cell power support and nominal targeted service times of around 10 years.

Given their long-duration residence within the patient's body, it is common for auto-immune responses to motivate anatomical encapsulation of, at least portions of, the implanted device. This encapsulation response challenges the clinical capacity for device explantation at the end of service as simple lasso/snare-based catheter systems cannot readily combat the increased device/physiology entanglement as compared to acute retrieval/explantation needs.

Presently, companies that offer implantable leadless pacer systems have provided tooling support for device implantation and, at best, marginal or configurable support for acute device retrieval. Such acute device support typically leans on the use of the implantation catheter or the adaptation of such a system through its pairing with compatible retrieval snares. In scenarios serviced by such acute device support, no gross autoimmune patient response is involved which means there are no substantially confounding anatomical conditions in effect to mandate compensatory procedural manipulations and/or mechanical interactions with the implant. As such, managing the device encapsulation, associated with chronically implanted conditions, has been left out of scope for catheter-based systems made available by leadless device manufacturers and thus represents an undersupported need.

This undersupport for chronic device explantation has arisen as a nearer term concern than many in the leadless pacer market might have hoped due to battery complications that have shortened product lifetimes. In some cases, the product lifetimes have been reduced to less than half of the nominal 10 year duration—a reduction sufficient enough to allow for encapsulation, creating special needs for appropriate management.

There is no consensus on how to best manage implants once their primary cells are no longer able to provide therapy. Some clinicians have discussed leaving the devices in place and simply installing additional devices to enable replacement support therapies. Others have pointed to the possible use of acute explantation tools in cases where device encapsulation (in a given patient) is not severe.

In cases where an expired implant is not removed from the patient's body and additional devices are installed to provide replacement therapy, the patient accumulates an increasing quantity of in-body hardware as a function of time. This added hardware can interfere with the nominal operation of the heart through modified compliance of the heart tissue, and reductions in the overall functional heart chamber blood volume. As a result of the limited flexibilities available for placing long cylindrical devices in but a handful of locations in support of viable interfacing with the patient's conduction system, optimal placement of subsequent implants additionally proves challenging. Such conditions can lead to the further progression of disease states, compromised oxygenation of tissues in the periphery, and the need for higher pacing thresholds (and hence shorter service times) in subsequent implants. Further, it cannot be guaranteed that multiple devices “banging into one another” in the heart will not create complications for the new device, cause anchoring erosion, or other possible deleterious effects.

Using acute explantation catheters to try and perform chronic explantation demands alignment with optimal patient conditions. Such an approach is only viable if the patient's auto-immune response does not motivate substantial encapsulation. There is no known means to improve the likelihood that a patient would not have an encapsulation response. In addition, there is no readily known method for determining a device encapsulation state when the therapy support needs replacing. Such a shortcoming typically means that the clinician has to access the patient's vasculature and then “try out” the acute explantation tooling in hopes that they are lucky enough to have found a patient where gross implant encapsulation is not in effect.

International Publication No. WO 2017/065846 A1 describes an engagement subassembly of a catheter, for retrieving an implanted medical device. Particularly, the system may also include an outer sheath which includes a cutting distal-most edge.

Further, International Publication No. WO 2018/204753 A1 discloses catheter systems for delivery and retrieval of leadless pacemakers that may include a retriever that can include a pair of lumens through which snare wires are passed to tighten around a docking projection of the leadless pacemaker.

The present disclosure is directed toward overcoming one or more of the above-mentioned problems, though not necessarily limited to embodiments that do.

### SUMMARY

Based on the above, a problem to be solved by the present invention is to offer support for the separation of intracardiac medical devices, particularly leadless pacers, from surrounding physiologic encapsulation stemming from chronic implantation. Particularly, a further objective is to facilitate said support using a catheter-based system that introduces minimal (and ideally no) use-based complexity beyond systems used for implantation and/or acute explantation, and/or to facilitate said support by extracting as much of the surrounding device encapsulation to offer the cleanest heart wall interior and hence avoid confounding follow-on attempts to place new devices.

At least the above-stated problem is solved by a catheter system having the features of claim 1. Further embodiments are stated in the corresponding sub claims and are described below.

In one aspect, a catheter system for explanting an intracardiac medical device (also denoted as implant) is disclosed. The catheter system comprises:
a catheter having a distal catheter tip configured to surround the intracardiac medical device and an encapsulation that has grown around the intracardiac medical device, and
a deployable cutting element at the distal catheter tip.

Particularly, the present disclosure provides a catheter system that enables a means for viably and safely separating an intracardiac medical device along with its encapsulating tissue in-growth from surrounding patient anatomy.

Furthermore, even when deployed, the cutting element (or the individual cutting knife) is not exposed outside of the catheter tip/implant protector cup. Such a design facilitates the slicing of tissue only when it has been drawn internal to the catheter tip and when it is bound between knife edges and the implant body. This safety feature enables a safe means for severing such tissue without presenting undue risk for unintended patient harm.

According to an embodiment, the catheter system comprises a lasso for establishing a lasso-based (or tethered) linkage to the intracardiac medical device.

Furthermore, according to an embodiment, the catheter system comprises an alignment tube for tightening the lasso.

Furthermore, according to an embodiment, the catheter system comprises a ramping element (e.g. an alignment cup) on a distal end of the alignment tube for centering the catheter tip about the recaptured intracardiac medical device.

Furthermore, according to an embodiment, the distal catheter tip is formed by a protector cup configured to be moved along the lasso-based linkage to the intracardiac medical device to surround both the implant and its surrounding encapsulation.

Furthermore, according to an embodiment, the cutting element comprises a series of deployable cutting knives also denoted as knife-tipped features that when not deployed extend along a long axis of the catheter tip but when deployed point inward to sever encapsulation tissue. Particularly, for severing the encapsulation tissue, the catheter tip, particularly the entire catheter, is configured to be rotated about the long axis of the catheter tip. Therefore, particularly, the cutting knives are configured to cut the encapsulation tissue along a circumferential direction of the intracardiac medical device.

Furthermore, according to an embodiment, the respective cutting knife is formed by a finger having a preformed tip, wherein a preformed condition of the tip is suppressed by features internal to the catheter until the tip is intentionally deployed by the explanting clinician.

Furthermore, according to an embodiment, the catheter tip comprises an inner and an outer material layer forming said features internal to the catheter, wherein the respective cutting knife is configured to be moved between the outer and the inner material layer from a proximal position to a distal position along the long axis of the catheter tip with respect to the catheter tip so that the preformed condition of the tip is suppressed by the inner and the outer material layer.

According to an embodiment, the inner material layer comprises an opening such that when the inner material layer is rotated about the long axis of the catheter tip when the respective cutting knife resides in the distal position, the preformed tip of the respective cutting knife comes into the region of the opening of the inner material layer and is thereby released so that the tip can assume the preformed condition in which the tip of the respective cutting knife points inwards through said opening of the inner material layer.

Furthermore, according to an embodiment, the cutting element comprises grabber elements configured to be deployed to aid in the retention of removed encapsulation and/or the intracardiac medical device, particularly for removing the latter.

According to an embodiment, each grabber element resides on a different finger of the cutting element.

Furthermore, according to an alternative embodiment, each grabber element resides on an independent finger of the cutting element that does not form a cutting knife and is configured to be moved between the outer and the inner material layer from a proximal position to a distal position along the long axis of the catheter tip with respect to the catheter tip.

Furthermore, according to an embodiment, the respective grabber element is configured to be one of: always deployed during said movement from the proximal position to the distal position and/or upon said rotation; deployed prior to deployment of the cutting knives; deployed in coordination with the deployment of the cutting knives; deployed simultaneously to the deployment of the cutting knives; deployed after the deployment of the cutting knives.

Furthermore, according to an embodiment, the respective grabber element is a preformed bulged section of the respective finger, wherein the preformed condition of the respective grabber element is suppressed by the inner and the outer material layer when the corresponding finger is moved from the proximal position to the distal position, and/or wherein when the inner material layer is rotated about the long axis of the catheter tip when the respective cutting knife resides in the distal position, the respective grabber element comes into the region of the opening of the inner material layer and is thereby released so that the respective grabber element can assume the preformed condition in which the respective grabber element bulges inwards through said opening of the inner material layer.

Furthermore, according to an alternative embodiment, the respective grabber element is a bulged section of the corresponding finger, wherein the bulged section engages with an opening formed in the outer material layer when the respective finger is moved from the proximal position to the distal position and/or rotated about the long axis of the catheter tip when the respective finger resides in the distal position, and/or wherein the respective bulged section is pressed against the outer layer when the corresponding finger is retracted from the distal position to the proximal position so that the respective grabber element bulges inwards and is thereby deployed.

According to a further aspect, a method for explanting an intracardiac medical device is disclosed, wherein the method uses a catheter system according to the present disclosure, and wherein the method comprising the steps of:
recapturing the intracardiac medical device by using the lasso to establish a lasso-based (or tether-based) linkage to the intracardiac medical device and tightening the lasso using the alignment tube;
centering the catheter tip about the intracardiac medical device using the ramping element on the distal end of the alignment tube;
moving the distal catheter tip along the lasso-based linkage to the intracardiac medical device to surround both the intracardiac medical device and the encapsulation;
deploying the cutting element;
rotating the catheter tip to sever encapsulation tissue; and
retracting the catheter tip to remove the intracardiac medical device.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

In the following, embodiments as well as further aspects, features and advantages of the present invention are described with reference to the Figures, wherein

FIGS. 1A-1J show steps A to J of explanting an intracardiac medical device using an embodiment of the catheter system;

FIGS. 2A-2C show perspective views of different embodiments of cutting elements comprising cutting knives and optionally grabber elements;

FIG. 3 shows a depiction of a tip of a cutting knife of an embodiment of a catheter system that employs a format where only a series of cutting knives are deployed;

FIG. 4 shows a depiction a tip of a deployable cutting knife of an embodiment of a catheter system that employs a format where a grabber element of the cutting knife is always in a deployed state;

FIG. 5 shows a depiction of a tip of a cutting knife of an embodiment of a catheter system that employs a format where the respective cutting knife and the respective grabber element deploy simultaneously, in coordinated fashion; and

FIG. 6 shows a depiction a tip of a deployable cutting knife of an embodiment of a catheter system where the respective cutting knife deploys first and the respective grabber element deploys upon retraction of the tip of the cutting knife.

### DETAILED DESCRIPTION

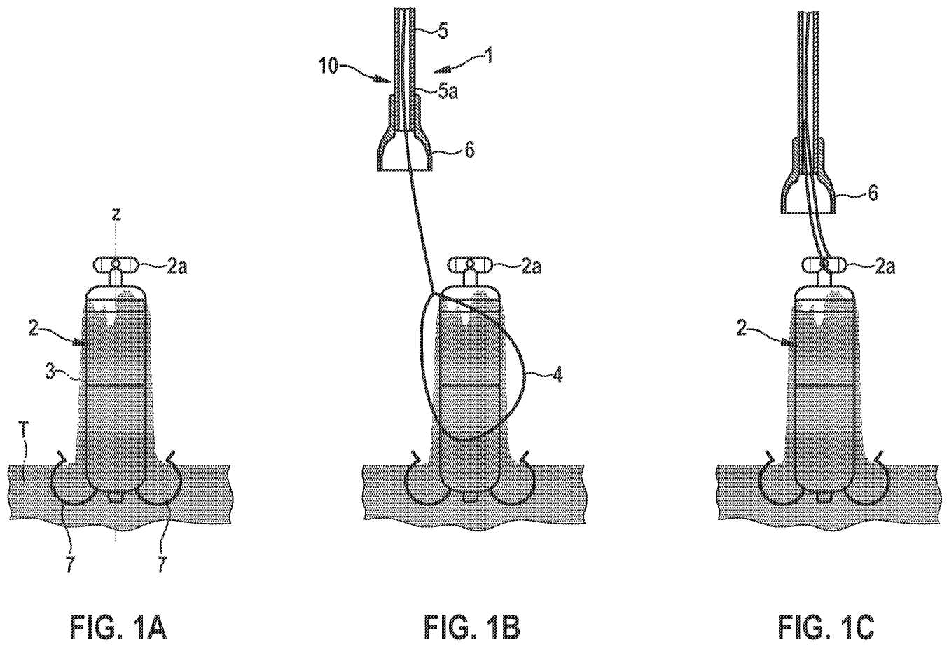

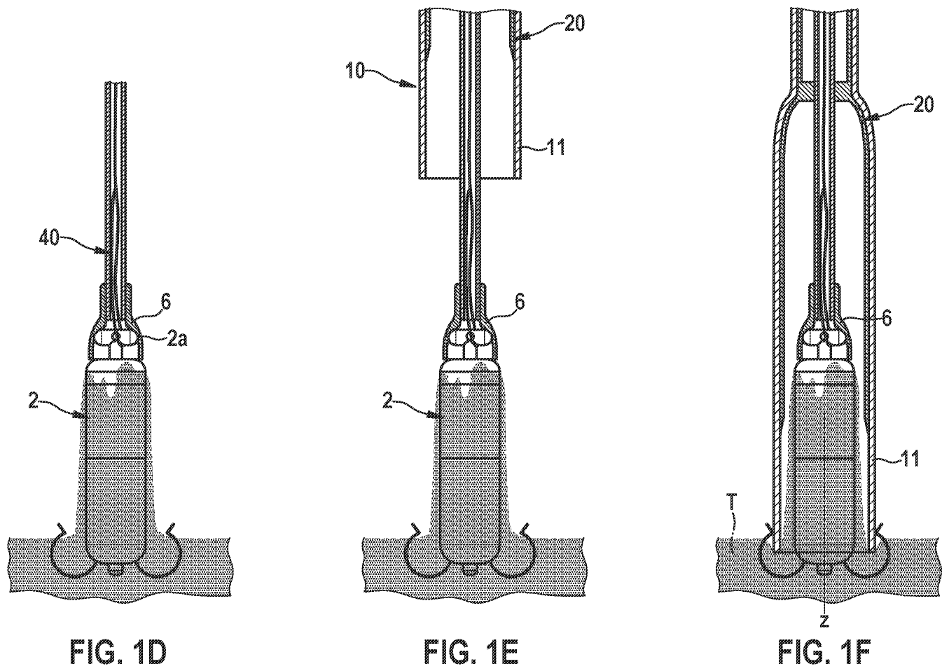

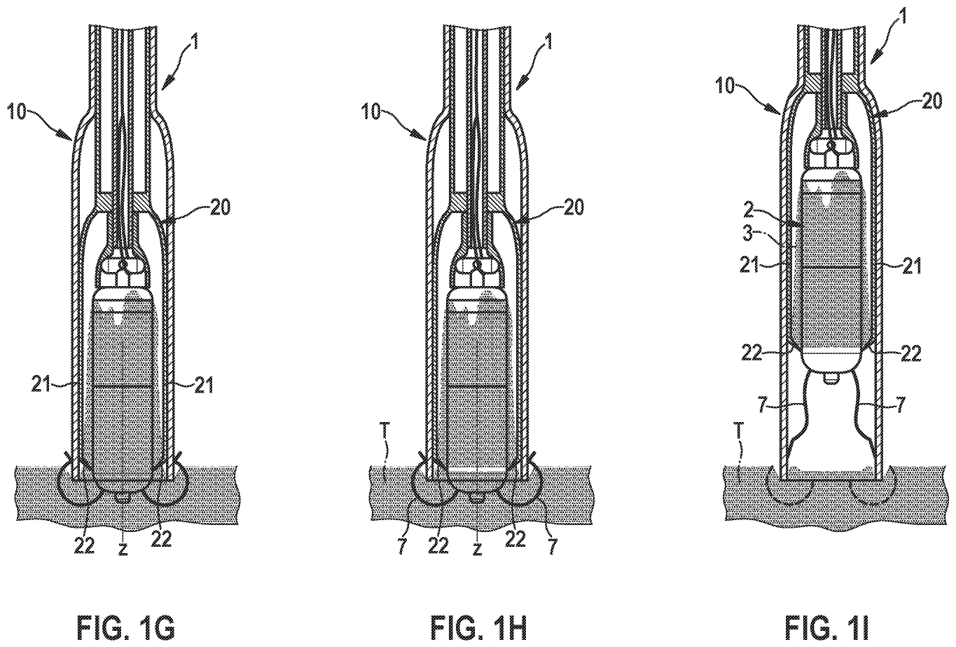

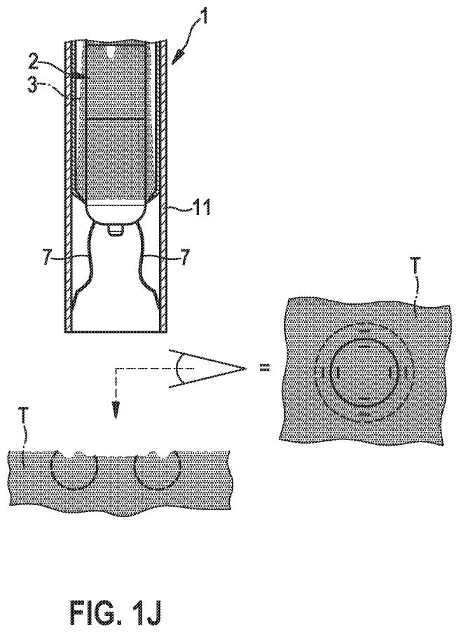

One embodiment employs a catheter-based system comprising a catheter 10 with a series of retractable cutting knives 21 at its distal tip 11 as e.g. shown in FIGS. 1A-1J. The catheter system 1 functions by first arranging a lasso 4 protruding out of an alignment tube 5 of the catheter system 1 to facilitate a lasso-based recapture of the proximal end 2a of the implanted intracardiac medical device (e.g. a leadless pacemaker) 2 that is anchored into tissue (e.g. a heart wall) T of the patient by means of a device anchor (e.g. tines 7). Furthermore, the lasso 4 is tightened using the affiliated cinch/alignment tube 5 (see steps B to D in FIGS. 1B-1D), e.g. by retracting the lasso into the tube 5. As a consequence a ramping element 6, here e.g. in the form of an alignment cup 6 arranged at the distal end 5a of the tube 5 receives said proximal end 2a of the implant 2 which allows to center the distal catheter tip 11 with respect to the implant due to the fact that the alignment tube 5 is slidably arranged in the catheter 10.

After having placed the alignment cup 6 on the proximal end 2a of the implant, an implant protector cup forming the distal tip 11 of the catheter 10 then tracks along the newly established lasso-based linkage 40 to the implant 2 to surround not only the implant 2 but also an encapsulation tissue 3 that has grown around the implant 2 (c.f. steps E to F in FIGS. 1E-1F). Because the protector cup 11 needs to surrounds both the implant 2 as well as its encapsulation tissue 3 its diameter has to be large enough.

Once the intracardiac medical device 2 is recaptured with the lasso 4 (steps B to D) and covered with the protector cup 11 (steps E to F), a series of retractable cutting knives 21 is deployed (cf. step G in FIG. 1G), the entire catheter system 1 is rotated X degrees about the long axis z of the catheter tip/proctor cup 11 to sever the encapsulation tissue 3 from e.g. the heart wall T (step H in FIG. 1H). The device anchor 7 is then separated from the heart wall T (step I in FIG. 1I), and the implant 2 along with its surrounding encapsulation tissue 3 are removed from the patient (step J in FIG. 1J, including an inset showing the remnants of the anchor site). It is to be noted that the depiction of FIGS. 1A-1J is drawn using a combination of “as seen” and cross-sectional views with intent to highlight design elements of the present disclosure.

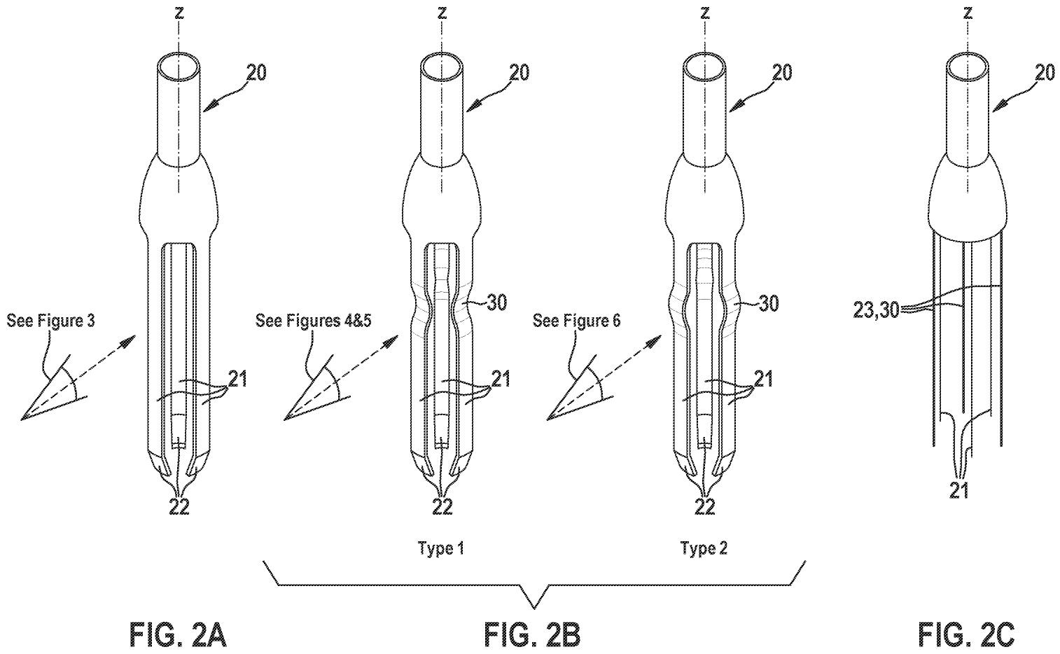

With the implant 2 reconnected to the catheter system 1, a structure 110, 111 internal to the catheter 10 facilitates the deployment of a series of knife-tipped features/cutting knives 21 (step G in FIG. 1G). These knife-tipped features 21 amount to a series of fingers 21 with preformed tips 22. The preformed condition of these tips 22 is however suppressed by features 110, 111 internal to the catheter 10 until the cutting knife tips 22 are intentionally deployed by the explanting clinician. FIGS. 2-6 further highlight further details associated with these features 110, 111 and the deployment of the cutting knifes 21 and optionally grabber elements 30.

Upon deployment, these knives 21 pierce into the encapsulation tissue 3 near the anchored terminus of the implant (e.g. leadless pacer) 2. Subsequent rotation of the entire catheter system by X degrees causes the tips 22 of the knives 21 to then slice around the bottom end of the encapsulation tissue 3, cleanly separating it from the heart wall T (step H in FIG. 1H). The amount of rotation necessary to fully cut around the perimeter of the implant 2 and effectively sever the encapsulation 3 is particularly dependent upon the number of knives 21 incorporated within the tip of the catheter's deployable cutting element 20.

The example embodiments shown in FIGS. 2A-2C show cutting elements 20 with 3 (knife-tipped) fingers 21 rotationally offset from one another by 120 degrees. As such, rotating the entire catheter system 1 or the catheter 10 by ⅓ of a complete rotation (360°) would notionally separate the encapsulation capsule 3 from the heart wall T (while a 4-finger system would only demand ¼th of a complete rotation, etc.) It should be noted that even when deployed, these knives 21 are not exposed outside of the implant protector cup 11. Such a design facilitates the slicing of tissue 3 only when it has been drawn internal to the catheter tip 11 and when it is bound between knife edges and the implant body. This safety feature enables a safe means for severing such tissue without presenting undue risk for unintended patient harm.

Once the capsule 2 has been separated, the cutting features internal to the catheter 10 are withdrawn distally in coordination with the cinch/alignment tube 5 while the implant protector cup 11 remains in a stable position (step I in FIG. 1I). This sequence permits the distal end of the implant protector cup 11 to serve as a stable counter force for safely withdrawing the implant's anchors 7 from the heart wall T without unintentionally inverting the heart chamber in divot-like fashion. In all shown embodiments within the present disclosure the knife edges remain pressed against the body of the implant 2 as the implant 2, the excised surrounding encapsulation 3, and the implant anchor 7 are pulled fully within the protector cup 11. By remaining pressed in place, they can ensure that the separated capsule 2 goes along for the ride and is successfully retained within the implant protector cup 11 rather than being left to enter passing blood flow where it could cause downstream physiologic complications. With the implant 2 and its surrounding encapsulation 3 separated from the heart chamber wall T, all are then removed from the patient leaving behind a clean surface that minimally interferes with follow-on device implant procedures (step J in FIG. 1J).

Details associated with the cutting elements 20 internal to the distal tip 11 of the catheter 10 are shown in FIGS. 2A to 2C.

FIG. 2A shows an embodiment where the cutting element 20 does not comprise grabber elements 30 as shown in FIGS. 2B and 2C. Such a cutting element 20 having cutting knives 21 (knife only format) is also shown in FIG. 1. Alternatively, also the cutting elements 20 according to FIGS. 2B and 2C can be used in the embodiment shown in FIG. 1.

The embodiments shown in FIGS. 2B and 2C further extend and enhance the functionality of these knives 21 with the inclusion of different types of grabber elements 30. Such grabbers 30 are intended to aid in the retention of the severed capsule 2 within the distal tip 11 of the explantation catheter 10. In other words, they offer added “hands” for helping hold onto the separated capsule 2 beyond what is offered by the having the tips 22 of each knife 21 maintaining contact with the body of the implant 2 during retraction into the implant protector cup 11.

Particularly, while FIG. 2A highlights a 3-D version of what was suggested within the sequence found in FIG. 1, the FIG. 2B offers an enhanced approach where grabber elements 30 are included on the cutting fingers 21 to better retain the excised implant encapsulation 3 while FIG. 2C suggests a variant of FIG. 2B where the grabbers 30 and knives 21 each reside on independent fingers 21, 23.

Again, the number of cutting knifes 21 within the distal tip 11 of the catheter 10 could be greater or fewer than the illustrative three shown in each of the depictions in FIG. 2. Additionally, the grabber elements 30 can be structured to point internal to the catheter 10 (Type 1 in FIG. 2B) or external to the catheter 10 (Type 2 in FIG. 2B) and as suggested in FIG. 2C, other embodiments could use independent fingers 21, 23 for the knives 21 and the grabbers 30.

As pointed to in FIGS. 2A to 2C, FIGS. 3 to 6 show details of a “gear shifting” mechanisms associated with the deployment of both knives 21 and grabber elements 30. For each figure, the top portion shows a stack-up of catheter layers 110, 111 in pictorial form as a reference for the first step shown in the middle sequence of operations. Further, the bottom half of each figure shows a cross section of critical steps to highlight deployment operations. In general the proposed movements, regardless of the specific cutting feature architecture amount to an extension process, a rotation of these features relative to the implant protector cup, a coordinated rotation of the entire catheter, and a retraction process.

In all the depictions shown, the blades stay deployed while all retraction processes occur as a means to enable proper retention of the removed capsule. This condition is not necessarily mandated but would require any in-tip “grabbers” to do all the work in holding onto the removed encapsulation if the tips were allowed to separate from the implant body as the device, the capsule, and the device anchor were withdrawn into the capsule.

Through the modification of the finger geometries within the cutting apparatus and the cutouts or openings 110a, 111a within the stacked material layers 110, 111 found at the distal tip 11 of the catheter 10, the behaviors of the knives and grabbers can differ.

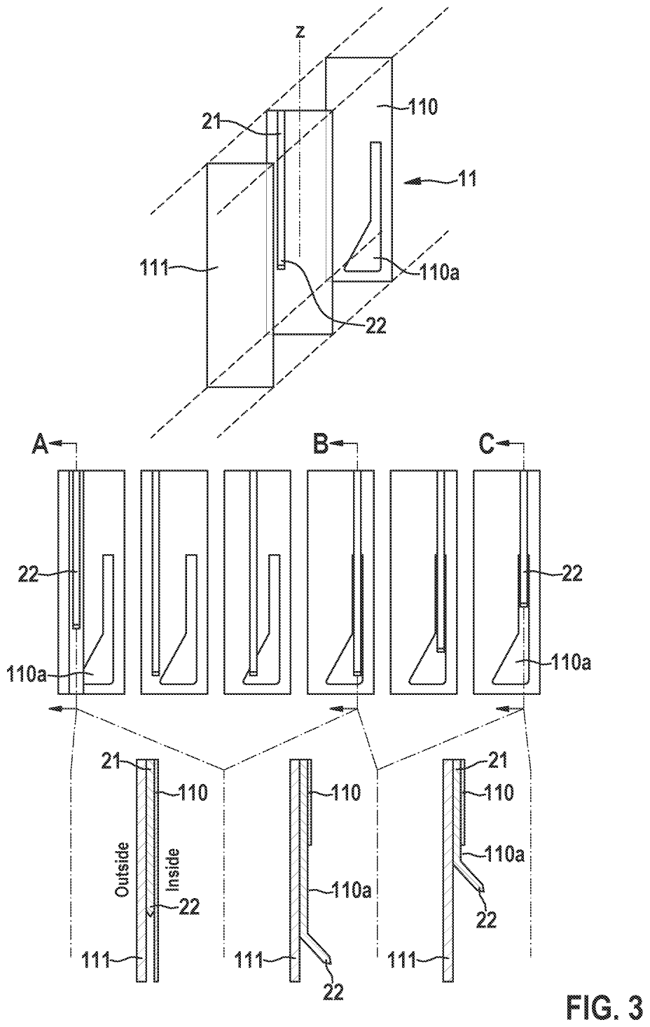

FIG. 3 shows a depiction of the tip 22 of a catheter system 1 that employs a format where only a series of knives 21 are deployed. In the FIGS. 3 to 6 only one cutting knife is shown for reasons of simplicity. In this and all subsequent figures, the top illustration offers an exploded pictorial view of the first step in the left to right sequence found within the middle portion of the figure while the lower portion highlights a handful of key cross sections.

Particularly, according to FIG. 3, the catheter tip/protector cup 11 comprises an inner and an outer material layer 110, 111, wherein the respective cutting knife 21 is configured to be moved between the outer and the inner material layer 110, 111 from a proximal position to a distal position along the long axis z of the catheter tip 11 with respect to the catheter tip 11 so that the preformed condition of the tip 22 of the respective cutting knife 21 is suppressed by the inner and the outer material layer 110, 111 (cf. step A in FIG. 3).

Furthermore, as shown in FIG. 3, the inner material layer 110 comprises a cutout or opening 110a such that when the inner material layer 110 is rotated about the long axis z of the catheter tip 11 when the respective cutting knife 21 resides in the distal position, the preformed tip 22 of the respective cutting knife 21 comes into the region of the opening 110a of the inner material layer 110 and is thereby released so that the preformed tip 22 of the respective cutting knife 21 can assume the preformed condition in which the preformed tip 22 of the respective cutting knife points inwards (cf. step B in FIG. 3). Particularly, step C of FIG. 3 shows that the tip 22 maintain the deployed state upon retraction of the finger 21.

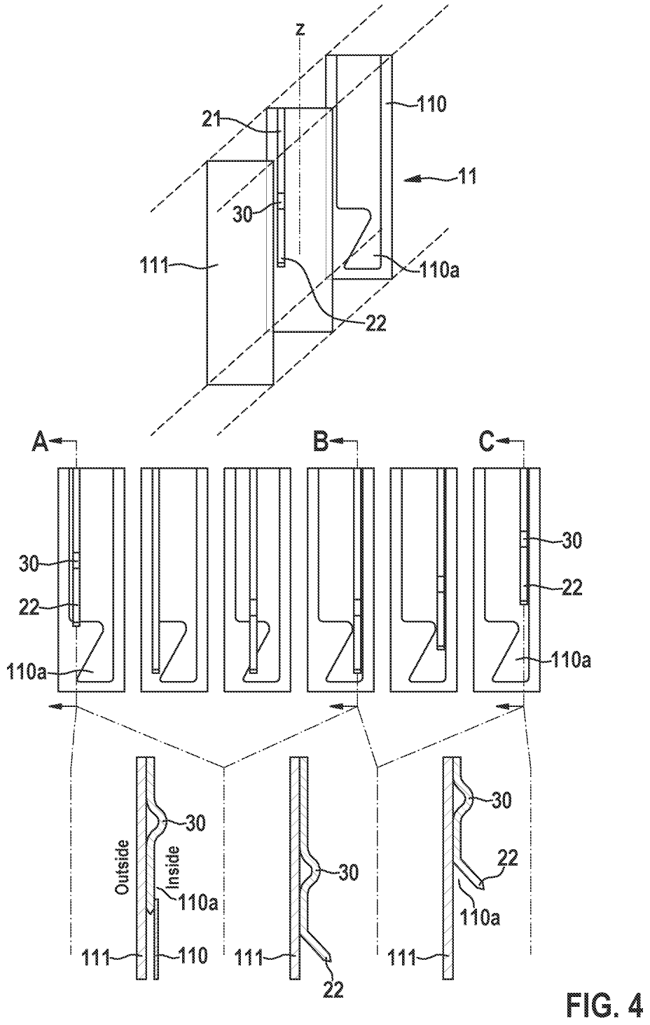

Furthermore, FIG. 4 shows a modification of the embodiment shown in FIG. 3, wherein here the respective cutting knife/finger 31 comprises a grabber element 30 in the form of a bulged section 30 of the finger that is always not suppressed by the inner material layer but extends through the opening 110a of the inner layer 110 and is therefore constantly deployed.

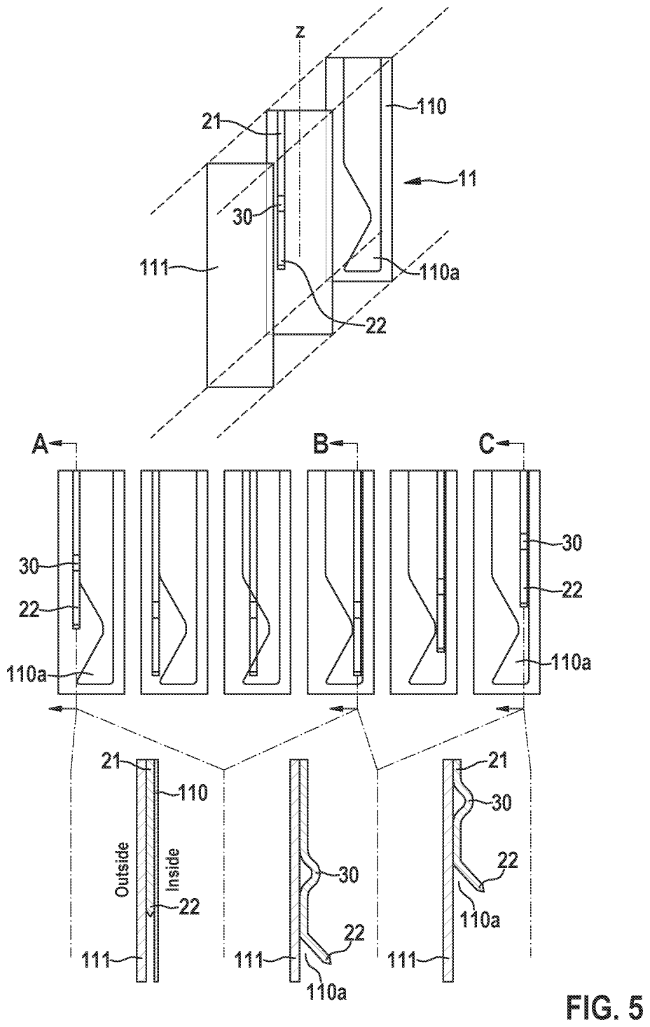

In contrast, FIG. 5 shows an embodiment where the respective grabber element 30 and knife 21 deploy simultaneously. Here, the respective tip 22 and the grabber element 30 formed on the finger 21 of the respective tip are both preformed and suppressed during moving the finger 21 along the long axis z from the proximal position to the distal position (cf. step A in FIG. 5). Upon rotation of the inner layer 110 about the long axis z, both structures 22, 30 simultaneously engage with the opening 110a of the inner material layer 110 and are thus allowed to deploy (step B in FIG. 5). This deployment is maintained upon retracting the respective finger 21 according to step C of FIG. 5.

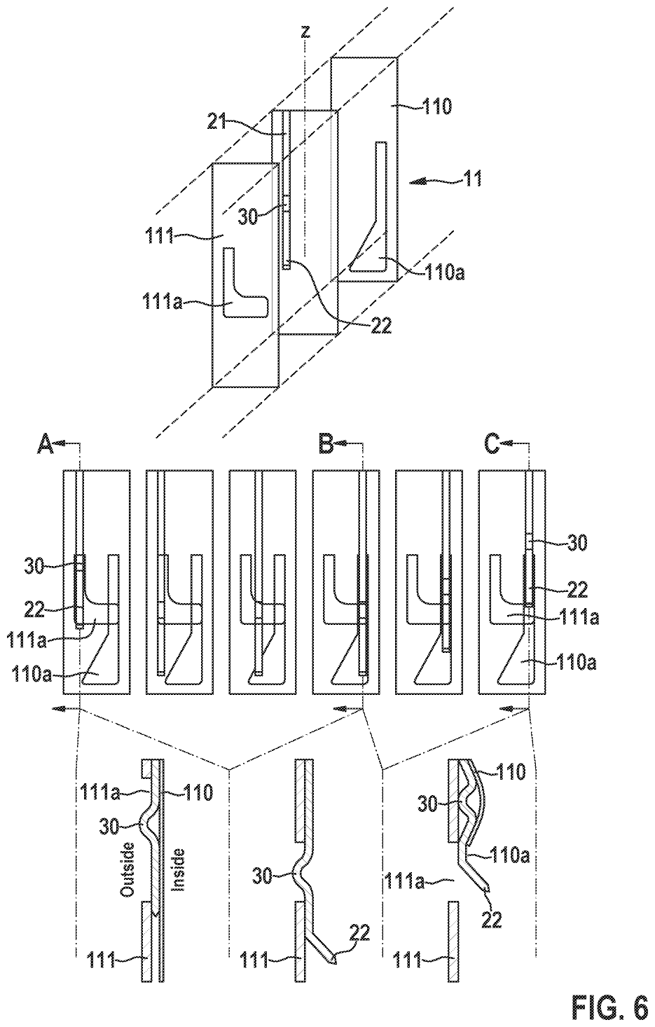

Finally, FIG. 6 shows an embodiment where the knife 21 deploys followed by the grabber element 30. Here, particularly, while the respective preformed tip 22 deploys as before upon rotation (cf. step B of FIG. 6) of the inner material layer 110 about the long axis z after the finger 21 has assumed its distal position, the grabber element 30 is a bulged section of the corresponding finger 21, wherein the bulged section 30 engages with an opening 111a formed in the outer material layer 111 when the corresponding finger 21 is moved from the proximal position to the distal position and/or rotated about the long axis z of the catheter tip 11 when the corresponding finger 21 resides in the distal position. Particularly, as indicated in step C of FIG. 6, the bulged section 30 of the corresponding finger 21 is pressed against the outer material layer 111 when the corresponding finger 21 is retracted from the distal position to the proximal position so that the respective grabber element 30 bulges inwards and is thereby deployed to hold the encapsulation 3/implant 2.

According to further aspects, the catheter system can comprise one or several of the following features, either alone or in any combination with each other:
A deployable cutting element at the distal tip of the explantation catheter.
Deployment of such cutting elements being facilitated via structuring of channels and cutouts within stacked material layers at the distal tip of the catheter.
Said cutting element comprising a preformed structure with a series of knife-like elements that when not deployed remain nominally conformal to the long axis of the catheter tip but when deployed point inward to sever encapsulation tissue withdrawn internal to the distal end of the catheter.
Said cutting elements facilitating cutting behaviors by creating a shearing surface between the blades of the knives and the exterior of the leadless pacer housing withdrawn into the tip of the catheter.
Each knife-like element residing at the distal end of a finger-like extension.
In enhanced formats further including a series of “grabber” elements within the distal tip of this deployable cutting element.
Said “grabbers” offering a capacity to aid in the retention of any encapsulation removed from the heart wall.
Said “grabbers” residing on the same fingers as the cutting fingers in some instances.
Said “grabbers” residing on independent fingers in other instances.
Said “grabbers” being in some cases always deployed and in others deployable.
Deployment of any deployable “grabbers” being facilitated via structuring of channels and cutouts within stacked material layers at the distal tip of the catheter.
Said structuring of channels and cutouts within the stacked material layers at the distal tip enabling “grabber” deployment, prior to, in coordination with (i.e. simultaneously), or after the deployment of the in-system knives.
A distal catheter tip sized sufficiently to surround both the leadless pacer targeted for explantation as well as the implant's surrounding encapsulation capsule/tissue.
A means for reliably centering this cutting catheter tip about the recaptured implant through a ramping element (i.e. the alignment cup) on the distal end of the cinch/alignment tube.
A means for enabling severing of encapsulation without instating unsafe tension, compression, or torque at the anchoring sight through the rotation of the protector cup relative to a fixed position cinch/alignment cup.
A means for presenting a stable counterforce at the anchor site to facilitate tine-based anchor removal without instating unsafe forces on the heart.
A means for removing the encapsulation capsule such that it does not present an “empty sock” tube of tissue once the implant has been removed.
A means for avoiding the freeing of capsule “debris” into freely circulating blood (i.e. extracting the excised capsule from the patient's body).

Potential advantages of the inventive solution:
The catheter system may be used as a means for removing any leadless system if sized appropriately.
If an “old” device can be removed, the “new” device does not have to worry about possible problematic interactions, mechanical or otherwise, and the clinician accesses a much broader selection of available anchoring sites.
New patient populations may even become accessible with this offering in place as placement in younger patients, for example, becomes more palatable from the vantage of subsequent therapy support.

The features disclosed in regard with the system may also apply to a method for explanting an intracardiac pacing system and vice versa.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Catheter system explanting an intracardiac medical device comprising:
a catheter having a distal catheter tip to surround the intracardiac medical device and an encapsulation tissue that has grown around the intracardiac medical device, and
a deployable cutting element at the distal catheter tip.

2. The catheter system according to claim 1, wherein the catheter system comprises a lasso for establishing a lasso-based linkage the intracardiac medical device.

3. The catheter system according to claim 2, wherein the catheter system comprises an alignment tube for tightening the lasso.

4. The catheter system according to claim 3, wherein the catheter system comprises a ramping element on a distal end of the alignment tube for centering the catheter tip about intracardiac medical device.

5. The catheter system according to claim 2, wherein the distal catheter tip is a protector cup configured to be moved along the lasso-based linkage to the intracardiac medical device to surround the intracardiac medical device and the encapsulation tissue.

6. The catheter system according to claim 1, wherein the cutting element comprises deployable cutting knives that when not deployed extend along a long axis (z) of the catheter tip but when deployed point inward to sever encapsulation tissue.

7. The catheter system according to claim 6, wherein the respective cutting knife is formed by a finger having a preformed tip, wherein a preformed condition of the tip suppressed by the catheter until the tip is deployed.

8. The catheter system according to claim 6, wherein the catheter tip comprises an inner and an outer material layer, wherein the respective cutting knife is configured to be moved between the outer and the inner material layer from a proximal position to a distal position along the long axis (z) of the catheter tip with respect to the catheter tip that the preformed condition of the tip of the respective cutting knife is suppressed by the inner and the outer material layer.

9. The catheter system according to claim 8, wherein the inner material layer comprises an opening such that when the inner material layer is rotated about the long axis (z) of the catheter tip when the respective cutting knife resides in the distal position, the preformed tip of the respective cutting knife comes into the region of the opening of the inner material layer and is thereby released so that the preformed tip of the respective cutting knife can assume the preformed condition in which the preformed tip of the respective cutting knife points inwards.

10. The catheter system according to claim 1, wherein the cutting element comprises grabber elements configured to be deployed to hold removed encapsulation tissue and/or the intracardiac medical device, particularly for removing the latter.

11. The catheter system according to claim 7, wherein each grabber element resides on one of the fingers.

12. The catheter system according to claim 8, wherein each grabber element resides on a finger of the cutting element that does not form a cutting knife and is configured to be moved between the outer and the inner material layer from a proximal position to a distal position along the long axis (z) of the catheter tip with respect to the catheter tip.

13. The catheter system according to claim 10, wherein the respective grabber element is configured to be one of: always deployed; deployed prior to deployment of the cutting knives; deployed in coordination with the deployment of the cutting knives; deployed simultaneously to the deployment of the cutting knives; deployed after the deployment of the cutting knives.

14. The catheter system according to claim 8, wherein the respective grabber element is a preformed bulged section of the corresponding finger, wherein the preformed condition of the respective grabber element is suppressed by the inner and the outer material layer when the corresponding finger is moved from the proximal position to the distal position, and/or wherein when the inner material layer is rotated about the long axis (z) of the catheter tip when the corresponding finger resides in the distal position, the respective grabber element comes into the region of the opening of the inner material layer and is thereby released so that the respective grabber element can assume the preformed condition in which the respective grabber element bulges inwards.

15. The catheter system according to claim 8, wherein the respective grabber element is a bulged section of the corresponding finger, wherein the bulged section engages with an opening formed in the outer material layer when the corresponding finger is moved from the proximal position to the distal position and/or rotated about the long axis (z) of the catheter tip when the corresponding finger resides in the distal position, and/or wherein the bulged section of the corresponding finger is pressed against the outer material layer the corresponding finger is retracted from the distal position to the proximal position so that the respective grabber element bulges inwards and is thereby deployed.
